(19) United States
(12) Patent Application Publication
(10) Pub. No.: US 2009/0326399 A1
(43) Pub. Date: Dec. 31, 2009

(54) MAGNETIC FIELD SENSOR, SYSTEM AND METHOD FOR DETECTING THE HEART BEAT RATE OF A PERSON IN A VEHICLE, AND SYSTEM AND METHOD FOR DETECTING FATIGUE

(75) Inventors: Juan Jose Barrero Batalloso, Cerdanyola del Valles (ES); Marc Fabregas Bachs, Cerdanyola del Valles (ES); Lluis Miquel Martinez Garcia, Cerdanyola del Valles (ES)

Correspondence Address: CANTOR COLBURN, LLP, 20 Church Street, 22nd Floor, Hartford, CT, 06103, US

(73) Assignee: ADVANCARE, S.L., Hospitalet de Llobregat (ES)

(21) Appl. No.: 12/298,207

(22) PCT Filed: Apr. 25, 2007
(86) PCT No.: PCT/EP07/54044

§ 371 Date: Jul. 1, 2009

(30) Foreign Application Priority Data
Apr. 26, 2006 (EP) PCT/EP2006/003858

(51) Int. Cl. A61B 5/0402

(52) U.S. Cl. 600/509; 600/508

(57) ABSTRACT

A system for detecting the heart beat rate of a person in a vehicle, comprising: at least one magnetic field sensor (11, 12) mounted inside the vehicle in a position close to a person's seat in the vehicle; and signal processing circuitry (2, 13) arranged to receive an output signal from said at least one magnetic field sensor, and to extract from said output signal data indicative of a heart beat rate. The invention also relates to a system for fatigue detection, and to the corresponding methods.

### FIELD OF THE INVENTION

The invention relates to the monitoring of physical parameters of a person, such as a driver of a vehicle. More specifically, the invention relates to the monitoring of the heart beat rate or cardiac frequency of a person, and also to fatigue detection based on a detected heart beat rate.

The invention also relates to a magnetic field sensor useful, for example, for detection of the heart beat rate.

### STATE OF THE ART

Traditionally, the heart beat rate (also referred to as heart rate (HR) in this present text) or cardiac frequency has been monitored mechanically, for example, by sensing the pulsations of a blood vessel, and electronically using electrodes attached to the body. Also, as the electrical pulses corresponding to the heart beat also generate a low frequency magnetic field (equivalent to a dipolar magnetic moment of a few μAm²), techniques have been developed for measuring heart beat related parameters magnetically. Basically, these techniques are based on SQUID magnetometry, and have proven to be useful for medical so-called magnetocardiography (MCG) (cf., for example, U.S. Pat. No. 6,745,063). However, SQUID magnetometry requires the use of complex and bulky devices and involves cryogenics. Some authors (Nathan A. Stutzke, et al., “Low-frequency noise measurements on commercial magnetoresistive magnetic field sensors”, JOURNAL OF APPLIED PHYSICS 97, 10Q107 (2005)) have analysed the use of magnetoresistive field sensors, including spin valves, and concluded than the detectivity is too low to make such detectors useful for MCG applications.

Mapps, D. J., “Remote magnetic sensing of people”, SENSORS AND ACTUATORS A (PHYSICAL), ELSEVIER SWITZERLAND, vol A106, no. 1-3, 15 Sep. 2003, pages 321-325 (XP002416449, ISSN: 0924-4247) generally relates to the remote sensing of people and focuses on SQUID devices and measurements of MCG inside a magnetically shielded room. However, one of the measurements (inside a shielded area) is performed with a fluxgate sensor.

As mentioned above, SQUID (Super conducting QUantum Interference Device) sensors require cryogenic temperatures, imply more complexity, higher costs and, also, due to the cryostat wall, a gap of a few centimetres between the chest (or back) and the sensors. As the magnetic field generated by the heart is mainly dipolar and, thus, decreases a lot with distance, the SQUID sensors should need to detect a field in the order of tens of pT (picoteslas), something extremely difficult in an environment such as a motor vehicle.

The so-called fluxgate, also described in, for example, R. H. Koch and J. R. Rozen, “Low noise fluxgate field sensors using ring and rod core geometries”, Applied Physics Letters, Mar. 26, 2001, Volume 78, Issue 13, pp. 1897-1899, can be used, together with suitable electronics (such as the one described in S. Takeuchi and K. Harada, “A RESONANT-TYPE AMORPHOUS RIBBON MAGNETOMETER DRIVEN BY AN OPERATIONAL AMPLIFIER”, IEEE TRANSACTIONS ON MAGNETICS, VOL. MAG-20, NO. 5, SEPTEMBER 1984, pp. 1723-1725), for detecting low magnetic fields. However, when not in a magnetically shielded environment, the measurement of MCG or heart beat rate magnetic signals is difficult, due to the presence of other interfering magnetic field sources. The Earth's magnetic field, for example, has vertical and horizontal components in the range of tens of μT (microteslas). Also, the existence of soft ferromagnetic objects can imply local disturbances and contributions (which can be significant within, for example, a motor vehicle).

Measuring the cardiac frequency or heart beat rate (also known as heart rate, HR) can be a bit less challenging than obtaining a full MCG measurement, since one can focus on the MCG peaks and disregard the details of the rest of the QRS curve. However, the level of the signal to be measured, close to the chest or the back of a person, will still be in the 1 nT (nanoTesla) range.

On the other hand, in the field of automotive vehicles there has been an increasing interest in the detection of parameters useful for determining the physical state of the driver of the vehicle, for example, so as to detect a medical emergency condition or simply to detect fatigue of the driver. For example, U.S. Pat. No. 6,946,965 describes a prior art driver fatigue detector basically based on the detection of a lack of reaction of the driver to a stimulus, and EP-A-1477117 describes a driver fatigue detector based on the detection of a blinking motion of the eyelids of the driver.

JP-A-11-151230 discloses a driver state measuring instrument which detects a physical condition of the driver using electrodes. The heart beat rate is detected by using electrical contacts on the steering wheel, and the variability of the heart rate is analysed to determine the physical condition of the driver. However, problems occur when the driver, for example, removes a hand from the steering wheel.

WO-A-2004/100100 generally relates to the detection of a condition of distress by measuring physical parameters of a person in a vehicle. As an example, it refers to “magnetic means arranged as a resonant circuit, said magnetic means being conceived to induce an oscillating magnetic field in a body volume”. That is, a kind of sensor is referred to that generates an oscillating magnetic field and measures how the body modulates or changes this magnetic field in order to, for example, obtain blood flow related information. Here, there is no reference to any magnetic field sensor, that is, to any sensor measuring at least one component of the magnetic field vector at the position in space where the sensor is located (such as, for example, a fluxgate, spin valve, or magnetoresistive sensor).

A sensor measuring one or more components of the magnetic field vector will directly measure a parameter directly related to the electrical pulse generated by the heart, which implies a very robust measurement of the heart beat rate, because the magnetic field generated by the heart is in the order of 1000 times larger than the magnetic fields generated by other electrical currents in the human body.

On the contrary, the blood flow measurement is an indirect measurement, based on body bioimpedance variation as a result of the heart beats. Nevertheless, the bioimpedance measurement is very noisy and full of artefacts (related to, for example, the blood composition, body composition—hydration, fat, etc.—or movement of the person under test) which will be superposed to the heart rate related information, making it difficult to reliably extract said information. That is why the heartbeat measurement by bioimpedance is difficult and, therefore, it has been ruled out as a diagnostic tool for the medical community.

Additionally, the sensor described in WO-A-2004/100100 is generating an oscillating electromagnetic field over the body which can affect the person subjected to it, and even be harmful to people with electronic implants (such as a pacemaker). The magnetic field sensors, as the ones described in the present application, are just measuring the magnetic field generated by the monitored person, without emitting electromagnetic waves or radiation to him or her.

The analysis of the heart rate variability (HRV) is a known technique used to evaluate the cardiovascular changes produced during the awake-asleep cycle (cf. Task Force of The European Society of Cardiology and The North American Society of pacing and Electrophysiology, “Heart rate variability. Standards of measurement, physiological interpretation, and clinical use”, Guidelines, European Heart Journal 1996; 17: 354-381).

Two major objective changes of the HRV between the awake and asleep states are well described in the literature:

(a) the heart rate (HR) decreases between 10 and 20%, between the moment when the person is completely awake and the moment when the person is completely asleep, but before reaching the first REM stage of the sleep;

(b) there are changes in the HRV (for example, the ratio between the spectral power density of the LF band (0.04-0.15 Hz) and the HF band (0.15-0.4 Hz), LF/HF, decreases 50-70%) between the moment when the person is completely awake and the moment when the person is completely asleep, but before reaching the first REM stage of the sleep. (Cf.: Melinda Carrington, Michelle Walsh, “The influence of sleep onset on the diurnal variation in cardiac activity and cardiac control”, Journal of Sleep Research (2003) 12, 213-221; M. Nakagawa, T. Iwao, “Circadian rhythm of the signal averaged electrocardiogram and its relation to heart rate variability in healthy subjects”, Heart (1998) 79, 493-496; Andrzej Bilan, Agnieszka Witczak, “Circadian rhythm of spectral indices of heart rate variability in healthy subjects”, Journal of electrocardiology (2005) 38, 239-243; Helen J. Burgess, Jan Kleiman, “Cardiac activity during sleep onset”, Psychophysiology (1999) 36, 298-306).

However, the literature focuses on the behaviour during the two states, but not on the transition between these states.

### DESCRIPTION OF THE INVENTION

A first aspect of the invention relates to a system for detecting the heart beat rate (that is, the cardiac frequency) of a person in a vehicle (for example, the driver or a passenger). The system comprises:

at least one magnetic field sensor mounted inside the vehicle in a position close to a person's seat in the vehicle; and

signal processing circuitry arranged to receive an output signal from said at least one magnetic field sensor, and to extract from said output signal data (such as specific values, or a signal indicative of said values) indicative of a heart beat rate.

In this document, the expression “magnetic field sensor” is intended to designate a sensor that is suitable for measuring at least one component of the magnetic field vector at a position in space where the sensor is located.

The use of one or more magnetic field sensors makes it possible to overcome the disadvantages involved with prior art systems requiring a direct contact between the user and the equipment used to measure the heart beat rate (for example, direct ohmic contact necessary for obtaining ECGs).

Said at least one magnetic field sensor can, for example, be mounted in a seat belt for the person in the vehicle, or in the person's seat.

Said at least one magnetic field sensor can comprise at least two magnetic field sensors, for example, two magnetic field sensors, both mounted in a seat belt for the person in the vehicle, both mounted in the person's seat, or one mounted in the person's seat and the other one mounted in the seat belt for the person. If one single magnetic field sensor is used, it can be a differential sensor comprising a plurality of “sub-sensors”, as described with more detail below.

Said at least two magnetic field sensors can be arranged to be placed substantially symmetrically with respect to the person's heart when the person is sitting in the vehicle, and/or said at least two magnetic field sensors can be arranged at different heights. The signal processing circuitry can be arranged to subtract an output signal from one of the magnetic field sensors from an output signal from another of said magnetic field sensor, so as to obtain a resulting signal less influenced by magnetic fields not originated by the heart of the driver.

The magnetic field sensors and the signal processing circuitry can be arranged so as to produce a subtraction of components of output signals from the magnetic field sensors that are related to external magnetic fields not originated by the heart of the driver, so as to obtain a resulting signal less influenced by magnetic fields not originated by the heart of the driver. This can, for example, be achieved by arranging two magnetic field sensors with their sensing axes in the same direction but opposed sense, and thereafter summing the output signal from these magnetic field sensors, using a summing circuit producing effective subtraction of signal components having different signs. Of course, the system must be arranged so as to prevent the components originated by the heart to be effectively subtracted from each other.

These arrangements make it possible to obtain a signal that can be used to detect the heart beat rate. It must be kept in mind that a motor vehicle is a difficult environment when one tries to perform low magnetic field measurements. The car itself has sources that generate magnetic fields (hard contribution) and has a lot of soft magnetic materials than distort the Earth's magnetic field (soft contribution). For devices using the Earth's magnetic field (high precision compasses, magnetic blind angle object detectors, etc.) located inside or near a car, these two contributions can be corrected. The standard procedure is based on turning the vehicle 360°, for example, a few complete turns on a roundabout, and plot the resultant in-plane field components on an X-Y plot; the resulting geometric figure is usually a non-centred ellipsoid. In a non-magnetic environment, the figure is expected to be a perfect circle with origin at (0.0). The deformation is due the soft magnetic contribution of the car and the off-centring is caused by the hard magnetic contribution. Correcting the geometrical parameters of the experimentally obtained off-centred ellipsoid, converting it to a centred circle, allows compensation of the dc-magnetic field contributions of the car (cf. for example the procedure detailed on page 4 of EP-B1-1414003).

The hard contribution comes mainly from the engine block and normally represents an equivalent magnetic dipolar moment of between 100 and 500 Am². The soft contribution will have a low frequency component due the relative movement between the motor vehicle and the magnetic north.

High electrical currents may also provide a significant contribution to the magnetic fields in the vehicle. Lights and signals represent the main low frequency contributions (the signals normally have a frequency of between 0.5 and 1 Hz).

The field measured by a magnetic field sensor inside the car can thus be determined by a plurality of dipolar magnetic sources and by the Earth's magnetic field. The contribution of each source to the total magnetic field normally varies with time. If the contribution of the heart of the driver is separated from the contribution from the other sources, the total magnetic field measured by a magnetic field sensor can be defined as:

Bᵢ=Bᵢ^{Heart}(t)+Bᵢ^{undesired}(t)==k(t)^{Heart}/(rᵢ−r_{Heart})³+Σⱼk(t)^{j}/(rᵢ−rⱼ)³+Bₑₐᵣₜₕ(t)

where the constants k(t) are proportional to the equivalent dipolar magnetic moment of every source, rᵢ−r_{Heart} the distance between the sensor and the heart, and rᵢ−rⱼ the distance between the sensor and the j-undesired source. Bₑₐᵣₜₕ(t) is the contribution of the Earth's magnetic field, which will be vary with time due the angular displacement of the car with respect to the magnetic north.

As the magnetic field is vectorial, the expression is valid for every magnetic field component. If two-axial or tri-axial magnetic field sensors are used, the expression should be applied to Bx, By and Bz.

If two magnetic field sensors are placed with their sensing directions arranged in parallel, the output signal from one of the sensors can be subtracted from the signal from the other sensor, thus subtracting the contributions to the magnetic field:

B₁−B₂=k(t)^{Heart}((r₁−r_{Heart})⁻³(r₂−r_{Heart})⁻³)+Σj(k(t)^{j}((r₁−rⱼ)⁻³−(r₂−rⱼ)⁻³))

If the sensors are placed closer to the monitored person than to the other sources, the first term will be magnified and the second will tend to zero. With a higher number of sensors, similar expressions can be obtained, even further reducing the contribution of the distant magnetic field sources.

Another problem is to provide a magnetic field sensor output signal have the lowest possible signal/noise ratio. Depending on the sensors used, the problem can be the low resolution (2.7 nT for a HMC1001 sensor) or the noise (10-30 pT/Hz^{-1/2} for an SDT sensor). In both cases, the sensors should be placed as close as possible to the heart. Better sensors (like fluxgates, improved magneto resistive sensors or spin valves) can allow a larger distance between sensor and heart.

The ideal position for a magnetic field sensor trying to measure magnetic signals from the heart in a controlled ambient is the opening of the fourth intercostal space (the location of ECG lead V2). Now, when trying to measure parameters of the heart of the driver of the vehicle, it can be more difficult to correctly position the sensors with respect to the heart; also, the specific physical characteristics of the driver can vary (height, corpulence . . . ). Placing two sensors separated several centimetres can help to reduce this problem (the heart will be close to one of the sensors, which will thus have a big contribution when subtracting the output signals; if the heart is placed “between” the sensors, the contribution of the heart to each output signal will be added when subtracting the output signals (as the sign of the contribution of the heart will be opposite for each sensor), whereas the undesired contributions (external magnetic fields) will probably have the same sign in each output signal).

In summary, the position of the sensors is an important aspect when the issue is to get a signal good enough to allow a heart beat rate to be determined.

The signal processing circuitry can comprise an amplifier such as a low-noise, low offset differential amplifier (also known as instrumentation amplifier) and, in some cases, a derivation circuit.

The signal obtained from the sensor has a very low amplitude, but is amplified by the amplifier. By using a differential amplifier with its inputs fed with the signals from the sensors, the amplification can be made without too much amplification of the noise present in these signals. The signal thus obtained corresponds to a magnetocardiogram (MCG), that is, it shows the magnetic variations caused by the beating of the heart.

The MCG signal is a differential signal, that is, a signal obtained by measuring the difference between the magnetic characteristics at two different positions (when two sensors are used, these two positions correspond to these two sensors). The signal obtained from one of the sensors is used as a reference value for the other signal, and both signals are used by the amplifier. Now, in some cases, there is an excess of fluctuations in the reference signal. In these cases, a derivative circuit can be used to provide a more stable reference signal out of the instable one, whereby this stable reference signal can be applied to the amplifier to improve amplification performance.

A filter circuit can be used to remove the parts of the MCG signal that correspond to information not related to the heart beat rate (heart rate, HR) and also to remove part of the noise that is still present at the output of the amplifier. Butterworth filters provide good results, but when linear responses (without signal distortion) are not required, Chebyshev filters or other types of filters with high attenuation of undesired signals can give the best results.

Thus, at the output of the filter, an electrical signal is obtained that basically contains the information indicative of the heart beat rate.

The filter circuit may not be strictly necessary. However, depending on the sensor used, the output signal from the amplifier can be rather noisy and, in most cases, the R peaks of the MCG wave (that is, its maximum values) will not be clearly visible, wherefore the filter module can be necessary. As explained above, the main function of the filter module is to reduce the noise characteristics and to amplify the MCG characteristics of the signal at the output of the amplifier, in order to make the R peaks clearly detectable (cf., for example, H. Dickhaus, et al., “CLASSIFICATION OF QRS MORPHOLOGY IN HOLTER MONITORING”, Proceedings of The First Joint BMES/EMBS Conference Serving Humanity, Advancing Technology, Oct. 13-16, 1999, Atlanta, Ga., USA; page 270; ©1999, IEEE).

Further, the signal processing circuitry can comprise an analogical-to-digital (A/D) converter for digitalizing the filtered signal, and a microprocessor unit arranged to mathematically treat the digitalized signal so as to extract the heart rate from the previously amplified and filtered MCG signal.

The signal processing circuitry can comprise fuzzy logic means for extracting said signal or data indicative of a heart beat rate from said resulting signal. These fuzzy logic means can be implemented in the above-mentioned microprocessor unit, and can comprise an algorithm for performing calculus to reject “false MCG peaks” in the (amplified, filtered and) digitalized signal (for example, due to a non-perfect behaviour of the filter).

Even after the filtering and processing mentioned above, the RR-interval obtained (that is, the time distance between the subsequent peaks of the MCG wave) can still have erroneous values if the sensor output is of bad quality (which is likely to be the case inside a motor vehicle). To get a coherent RR-interval, it can be necessary to process the values using medical rules (cf., for example, C. H. Kumar, et al., “A ROBUST R-R INTERVAL ESTIMATOR”, Proceedings RC-IEEE-EMBS & 14th BMESl; page 1995; ©01995, IEEE), i.e., monitoring the R-R evolution corresponding to the last heart beats detected and assuring that this evolution correspond with a typical beat-to-beat time trend (this can also be implemented in the above-mentioned fuzzy logic means, by suitably programming the microprocessor unit with the relevant medical rules).

These classification techniques can be used to perform a real time analysis aiming at obtaining reliable heart beat rate data, taking into account information on typical heart rate evolutions.

To avoid confusion at beat detection, predictive fuzzy logic can be used (for example, based on learnings from information obtained from previous beats and/or information on normal heart beat rate trends) to reject “anomalous beats” not eliminated by preceding parts of the system.

Thus, substantially correct beat time values (technically, the RR-intervals) can be obtained, and the successive values can be recorded in a memory. Even if no filtering module is used (for example, if the magnetic field sensors provide a sufficiently good and noise-less output), the anomalies (so-called “ectopic beats”) can be detected and automatically filtered using a suitable algorithm (cf., for example, George B. Moody, “SPECTRAL ANALYSIS OF HEART RATE WITHOUT RESAMPLING”; page 715; ©1993, IEEE).

Another aspect of the invention relates to a system for fatigue or drowsiness detection, which incorporates a system as described above and, further, a fatigue or drowsiness detector arranged to process the signal or data indicative of the heart beat rate to detect whether said data are indicative of fatigue or drowsiness of a person and, if said data are indicative of fatigue or drowsiness, to produce a fatigue or drowsiness warning event (for example, a visible and/or audible signal to alert a driver of the vehicle). In this context, we will use the term “fatigue” as a generic term, encompassing drowsiness.

The fuzzy logic means (if such means are incorporated) and the fatigue detector can, for example, be embodied in one single microprocessor unit.

The data processing for fatigue or somnolence detection can be performed in the same microprocessor unit as the one used for extracting the data concerning the heart beat rate, for example, by a special algorithm described below.

The accepted beat times (that is, heart rate indicative data such as “beat-to-beat” times, for the beats taken as “valid” beats in the above described process) can be stored in a memory buffer, typically storing at least 100 values. Once the buffer is full, at every beat, a new beat-to-beat time (or other heart rate indicative parameter) value can be stored into the buffer and the oldest one can be removed (that is, the buffer can operate as a classical FIFO buffer), whereby a new set of values can be obtained every time a new beat time value is recorded, approximately every second. Thereby, a first set of values can be ready for processing some seconds (for example, 100 seconds) after start of the monitoring.

The recorded heart beat rate sample (that is, for example, the sample comprising 100 subsequently recorded “beat-to-beat” times) can then be analysed to extract somnolence information, for example, for the purpose of detecting that a driver will fall asleep minutes before it happens, to avoid accidents. Different analysis can be performed, for example, time and frequency analysis.

For example, the fatigue detector can comprise software arranged to detect fatigue by establishing, based on the data indicative of the heart beat rate, at least one reference value and at least one current value, said fatigue detector being arranged to trigger a fatigue warning event (such as an alarm signal) when at least one current value deviates more than to a predetermined extent from the corresponding reference value.

The current value and the reference value can, for example, be values indicative of the data indicative of the heart beat rate (for example, values corresponding to an average of the registered heart beat rate data stored in a memory), or of the variability of the data indicative of the heart beat rate, or values corresponding to a spectral analysis of the data indicative of the heart beat rate (such as a ratio between a low frequency component and a high frequency component of a curve corresponding to the heart beat rate spectra).

Actually, said at least one current value and said at least one reference value can comprise a plurality of current values and reference values, selected from the group comprising
a current value and a reference value indicative of the data indicative of the heart beat rate (such as corresponding to an average of said heart beat rate data);
a current value and a reference value indicative of the variability of the data indicative of the heart beat rate; and
a current value and a reference value corresponding to a spectral analysis of the data indicative of the heart beat rate;

whereby said fatigue warning event can be arranged to be triggered when at least two of the current values deviate more than to a predetermined extent from the corresponding reference values.

These options will now be described more exhaustively.

A first possibility is temporal: the average beat time (time between subsequent R peaks) of the sample is lower (corresponding to a higher heart rate) when a person is awake than when the person is in a first sleep stage, corresponding to a drowsy state of the person (that is, when the person enters the drowsy state, there is a lower heart rate, and, thus, a longer beat-to-beat time). Monitoring the variation of the average beat time or heart rate, for example, taking the average of the last 50-500 beats, a somnolence parameter can be obtained. Using, for example, 100 samples, a threshold set between 5% and 15% of increase of the average beat time has been found to give rise to a drowsiness warning about 4 to 7 minutes before the driver falls asleep.

Another possible parameter for monitoring the drowsiness, using a temporal analysis, is based on the variability of the beat time over the sample. When a person is awake, he/she has a larger variability of the beat time interval (or the heart rate) than when he/she is at the initial sleep stage, that is, at the drowsy stage.

Beat time interval or heart rate variability can be calculated using statistical parameters over the sample of recorded data (for example, on the last 50-500 pieces of recorded data). The easiest way to implement this method may be using the standard deviation of the RR interval, or the square root of the mean squared differences of successive RR intervals. Using standard deviation, the variability of the HR or the beat time interval decreases around 40% between the awake and asleep states. Monitoring this parameter and its evolution in subsequent samples each comprising, for example, 100 pieces of data, a decrease of between 10% and 30% can be used to trigger a drowsiness warning 4 to 8 minutes before the driver falls asleep.

A third method is based on a frequency analysis. The spectral power density of the heart rate can be calculated at different bands, for example, at the so-called LF band (0.04-0.15 Hz) and HF band (0.15-0.4 Hz). The LF band is associated with the sympathic systems and the HF band with the parasympathic (or vagal) systems of the person. The LF/HF ratio, also known as the sympatho-vagal balance, is high when the person is awake (the symphatic systems, LF, prepares the body for activity) and low when the person is asleep (the parasympathic-vagal systems, HF, prepares the body for relax) (Cf.: John Trinder, Jan Kleiman, “Autonomic activity during human sleep as a function of time and sleep stage”, Journal Sleep Research (2001) 10, pp. 253-264).

The obtained and stored values concerning the RR intervals define a discontinuous tachogram. The (for example) last 50-500 values can be interpolated to obtain a continuous signal, so that it is possible to analyze its spectrum. A typical value for the interpolation can be 2 Hz. The spectrum can be calculated using different approaches like the FFT, Yule-Walker, Burg, or Lomb-Scargle methods. Then the spectral power density of the LF band (0.04-0.15 Hz) and HF band (0.15-0.4 Hz) can be calculated. The values can be recalculated every time a new beat time is entered into the memory, thus providing, for every new beat, an updated information on the variation of LF and HF spectral power density. Using the spectral power density calculated using the last 100 recorded samples, when the LF/HF decreases by for example 50% with respect to the initial awake state, a fatigue warning can be triggered; with the numbers mentioned above, this would typically take place between 4 to 6 minutes before the driver actually falls asleep.

Each one of the three drowsiness indicators may produce (depending, inter alia, on the person who is being monitored) a certain number or false alarms, especially if the thresholds are set to give the warning far in advance of the actual moment of falling asleep (that is, if low thresholds are used to trigger the alarm). To minimise the false alarms, a combination of two or more of the above mentioned parameters can be used. For example, standard variation and LF/HF ratio can be combined using an AND function (whereby the fatigue warning will only be issued when both parameters indicates danger of falling asleep).

The above-mentioned methods are only examples of methods that can be used to detect fatigue on the basis of a detected heart rate.

The person referred to above can be a driver of the vehicle, but also a passenger (it can be interesting to monitor also the state of the passengers, for example, so as to hold information on the passengers' physical state in the case of an accident).

Another aspect of the invention relates to a vehicle, including a system according to any of the preceding claims (including, for example, the respective sensors placed in one or more seats and/or seatbelts of the vehicle, for monitoring the heart rate of the driver and/or passengers).

A further aspect of the invention relates to a method for detecting the heart beat rate of a person in a vehicle. The method comprises the steps of:

arranging or disposing at least one magnetic field sensor inside the vehicle in a position close to a person's seat in the vehicle;

receiving an output signal from said at least one magnetic field sensor;

and extracting, from said output signal, data indicative of a heart beat rate.

What has been said about the system is also applicable to the method, mutatis mutandis.

For example, said at least one magnetic field sensor can be mounted in a seat belt for the person in the vehicle, and/or in the person's seat.

For example, said at least one magnetic field sensor can comprise at least two magnetic field sensors. These sensors can be mounted in the seat belt for the person in the vehicle, or in the person's seat, or one sensor can be mounted in the person's seat and the other one in the seat belt. Said at least two magnetic field sensors can be placed substantially symmetrically with respect to the person's heart when the person is sitting in the vehicle, and/or arranged at different heights.

An output signal from one of the magnetic field sensors can be subtracted from an output signal from another of said magnetic field sensor, so as to obtain a resulting signal less influenced by magnetic fields not originated by the heart of the driver.

Components of output signals from the magnetic field sensors that are related to external magnetic fields not originated by the heart of the driver can be effectively subtracted from each other (for example, by arranging the sensors with their sensing axes in the same direction but opposite sense, and then summing the measured signals), so as to obtain a resulting signal less influenced by magnetic fields not originated by the heart of the driver.

The data indicative of a heart beat rate can be extracted from said resulting signal, for example, by using fuzzy logic means.

The person can be a driver of the vehicle.

A further aspect of the invention relates to a method for fatigue detection, for detecting fatigue of a person in a vehicle, comprising the method described above, and further comprising the steps of processing the data indicative of a heart beat rate to detect whether said data are indicative of fatigue of a person and, if said data are indicate of fatigue, producing a fatigue warning event.

The processing of the data indicative of a heart rate can comprise the step of establishing, based on the data indicative of the heart beat rate, at least one reference value and at least one current value. The fatigue warning event can be triggered when at least one current value deviates more than to a predetermined extent from the corresponding reference value, that is, when the deviation between the current value and the reference value exceeds a pre-established threshold, for example, a threshold set to a fixed amount or a threshold expressed as a percentage of the reference value.

For example, at least one current value and reference value can be values indicative of the data indicative of the heart beat rate (for example, indicative of an average of said data), and/or at least one current value and reference value can be values indicative of the variability of the data indicative of the heart beat rate, and/or at least one current value and reference value can be values corresponding to a spectral analysis of the data indicative of the heart beat rate (for example, said current value and reference value can correspond to a ratio between a low frequency component and a high frequency component of a curve corresponding to the heart beat rate spectra).

Said at least one current value and said at least one reference value can comprise a plurality of current values and reference values, selected from the group comprising
a current value and a reference value indicative of the data indicative of the heart beat rate (for example, indicative of an average of said data);
a current value and a reference value indicative of the variability of the data indicative of the heart beat rate; and
a current value and a reference value corresponding to a spectral analysis of the data indicative of the heart beat rate. Thus, said fatigue warning event can be arranged to be triggered when at least two of the current values deviate more than to a predetermined extent from the corresponding reference values.

A further aspect of the invention relates to a magnetic field sensor suitable for, for example, performing MCG measurements and/or detecting the beat rate. This sensor, for detecting at least one component of the magnetic field vector at a position in space where the sensor is located, comprises

at least two cores (for example, annular cores), said cores being made up by an insulated amorphous magnetic wire, each core comprising a plurality of windings of said amorphous magnetic wire, said amorphous magnetic wire being arranged so that a current can flow through said wire so as to reduce a noise level of the sensor;

for each core, a primary winding arranged in a toroidal manner around said core, said primary winding comprising, for each of the cores, substantially the same number of turns around the core, said primary winding being arranged so that a time varying current (that is, any current which varies in time between two different current values, such as, for example, a sinusoidal, square-wave or triangular wave current) can be driven through said primary winding, said primary windings being connected in series so that the time varying current flowing through each primary winding is substantially the same;

for each core, a secondary winding arranged around the core, said secondary windings being connected in series and further being connected to an output terminal of the sensor, for providing an output signal at said output terminal.

For example, in the case of two cores, the secondary windings of the different individual cores are connected in series, so that if the winding sense of the primary winding of the two cores is the same, the secondary windings could be connected with an opposed winding sense, whereas if the winding sense of the primary winding of two cores is the opposite, the secondary windings could be connected having the same winding sense.

Thus, a “differential” sensor is obtained, the advantages of which can be understood from the description below.

In order to obtain an output signal from a magnetic field sensor that has a good quality, it can be important to reduce the contribution of external sources (such as the Earth's magnetic field or magnetic fields generated by the metallic parts of a vehicle or similar in which measurements are made) and to use a low-noise magnetic field sensor.

One way of reducing the contribution of external magnetic sources can involve the use of two separate magnetic field sensors, arranged in a differential manner, so that the contribution of the external sources can be reduced by subtraction of the contributions of said external sources to the output signals of each one of the magnetic field sensors, as outlined further above.

However, this does not solve the problem related to the noise generated by each sensor. Furthermore, both sensors need to be correctly calibrated, in order that the external sources affect them in substantially the same way. It has been proven that this tends to be a complex and costly procedure. This problem is, at least in partly, solved by using a sensor in accordance with the invention.

In this way, a differential magnetic field sensor (also know as magnetic gradiometer sensor) is obtained, that uses a core of a material implying a very low noise level.

The differential magnetic field sensor can, basically, comprise two or more individual sensors or “sub-sensors”. At least one of them can be placed at a location or position at which the component of the magnetic field to be measured is “comparatively high” or “strong” (for example, close to the source—for example, the heart—of the field to be measured, also referred to herein as the “target source”), and the other one can be placed at a location where the component of the magnetic field to be measured is lower (not so “strong”), such as a few centimetres away from the first sub-sensor. As the magnetic field is dipolar, the first sub-sensor will detect a substantial contribution from the target source and the other sub-sensor a smaller contribution. If other “external” sources (such as the “source” of the Earth's magnetic field, sources related to the motor of a vehicle, etc.) are comparatively far away (compared to the separation between the sub-sensors), their contribution to the total field at the position of each individual “sub-sensor” will be very similar. Thus, when the signals from the two individual sub-sensors are subtracted from each other (which can be achieved by a differential arrangement of the secondary windings, having regard to the sense of winding of the primary windings), the contribution of the external (“undesired”) sources to the output signal from the differential magnetic field sensor can be substantially cancelled, whereas the contribution of the target source will not be cancelled. According to the arrangement of the sub-sensors with regard to the target source, the field from the target source can be substantially stronger at one of the sub-sensors than at the other, or the sub-sensors can be arranged so that the detected fields from the target source “add up” on the output of the differential magnetic field sensor, instead of cancelling each other (for example, by arranging the sub-sensors so that the field originated by the target source at one of the sub-sensors has an opposed sense compared to sense of the field originated by the target source at the position of the other sub-sensor).

These approaches can be especially useful for the purpose of detecting the heart beat rate of, for example, a person situated in a vehicle, where the exact magnitude of the MCG signal is not important, but rather the way it changes in the short time range. Thus, a differential magnetic field sensor arrangement, ensuring that the different individual magnetic field sensors or sub-sensors making up the differential magnetic field sensor are substantially identical from a physical point of view, will reduce the complexity of the whole device and of its operation. This is achieved by the above arrangement, whereby a further reduced noise can be obtained by using amorphous magnetic wire as claimed.

The arrangement essentially corresponds to a differential magnetic field sensor made up by at least two differently coupled fluxgate sensors (or “sub-sensors”).

Basically, a fluxgate sensor is based on a magnetic core, formed by a material with a high magnetic permeability which changes substantially in accordance with a magnetic field applied to the core. This core is excited by a primary coil or winding which generates a magnetic field big enough to change the state of the core (from high permeability when the field is zero, to low permeability when the core is saturated by this high magnetic field). Several materials can be used, the most commonly known ones maybe being Permalloy (a nickel iron magnetic alloy; generically, the term refers to an alloy with about 20% iron and 80% nickel; Permalloy has a high magnetic permeability, low coercivity, near zero magnetostriction, and significant anisotropic magnetoresistance) and amorphous magnetic ribbons.

However, these materials have an intrinsic noise higher than desirable for applications like Heart Beat Rate detection.

However, according to the invention, an Amorphous Magnetic Wire (AMW) is used for building the cores of the differential magnetic field sensor of the invention. It has been described that applying a small direct current (DC) to this kind of wire, the external magnetic domains remain blocked and the noise level is reduced (cf. for example, the R. H. Koch and J. R. Rozen reference mentioned above).

The secondary windings can be arranged in different ways. For example,
the secondary winding can, for at least one of the cores, comprise a plurality of loops each of which surrounds the entire core, so that each loop extends over two substantially diametrically opposed portions of the core; or

the secondary winding can, for at least one of the cores, comprise at least two portions, one portion comprising a plurality of loops around a first perimetral portion of the core, and another portion comprising a plurality of loops around a second perimetral portion of the core, angularly displaced along the core with regard to said first perimetral portion (said second perimetral portion can, for example, be arranged substantially diametrically opposite said first perimeral portion); or

said secondary windings can comprise one single secondary coil wound so as to surround at least two of the cores, so that the same coil constitutes the secondary winding of each of said cores.

The way the secondary windings are wound around the cores should be related to the way the primary windings are wound so as to give rise to the differential output signal, as explained herein. For example, when one single “secondary” coil is used for the two cores, the primary windings should be connected in a way so that the current flows in opposite directions at the two cores, so as to give rise to the differential output signal. In this case, the winding sense of the secondary winding becomes irrelevant, as the use of a common coil itself implies a subtraction of the contributions.

The secondary windings can be interconnected so that when the same external magnetic field is applied to said at least two cores oriented in the same manner, the output signal is substantially zero. In this way, the contribution of the “distant” sources, such as the Earth's magnetic field, can be nulled, so that an output signal is obtained that is substantially only related to the target source, such as the heart of a monitored person, as long as the cores are placed so that both cores do not sense the magnetic field generated by said target source in the same way.

The (at least) two cores can be made up by one single amorphous magnetic wire, so that the current flowing through said, at least, two cores will be the same. Also, the primary windings of said at least two cores can be made up by one single conductive wire, so that the current flowing through said primary windings will be the same at said, at least, two cores. In this way, the degree of “identity” or “similarity” of the two sub-sensors will be improved.

The secondary windings can be made up by one single conductive wire having two terminals at which said output signal can be obtained.

The secondary windings can be serially connected so as to provide a differential output signal at least partly indicative of a difference between said component of the magnetic field at one of said cores and at another one of said cores.

This can help to obtain a differential output signal, that is, an output signal in which the contribution of an external (non-target) source to the magnetic field sensed at one of the cores or “sub-sensors” is subtracted from the contribution of said source sensed at the other core or “sub-sensor”, thus making it possible to distinguish a comparatively weak magnetic field originated by the target source.

The sensor can comprise at least two differentially coupled flux-gate sensors, each of said flux-gate sensors comprising one of said cores with the corresponding first and secondary windings. The arrangement disclosed above corresponds to a so-called fluxgate arrangement, which is well known to the skilled person (see, for example, Pavel Ripka, Review of Fluxgate sensors, Sensors and Actuators A 33, 1992, pp. 129-141).

The sensor can further comprise electronic circuitry so as to provide a differential output signal indicative of a magnetic field from a target source. The electronic circuitry can comprise means for treating and analysing an output signal at terminals of the secondary windings. Here, conventional (open loop) electronics for fluxgates can be used, or resonant (closed loop) ones such as the one disclosed in the S. Takeuchi and K. Harada reference mentioned above.

The electronics can comprise means for producing a DC current in said amorphous magnetic wire and a time varying current in said primary windings. The DC current or bias current could serve to reduce the noise level, as explained above (cf. also the Koch et al. paper cited above).

The sensor can be arranged to detect the heart beat rate of a person, for example, the sensor being arranged so that at least one of the cores is arranged substantially closer to the collarbone of the person than at least another of said cores. For example, at least a first one of said cores can be arranged within a distance of 10 cm from said collarbone, whereas at least a second one of said cores can arranged at a distance of at least 5 cm from the first one of said cores.

As an alternative or complement, a sensor of the invention can be arranged to detect the heart beat rate of a person, by being arranged so that at least one of the cores is arranged substantially closer to the left kidney of the person than at least another of said cores (for example, at least a first one of said cores can be arranged within a distance of 10 cm from the left kidney and at least a second one of said cores can be arranged at a distance of at least 5 cm from the first one of said cores).

Normally, when measuring a magnetic field for the purpose of establishing an MCG, the monitored persons is on a bed, and the magnetic field sensor or sensors are placed on the chest of the person under test, at the location of the ECG lead V2. Thus, the magnetic field monitored is mainly the component perpendicular to the chest, which in this case is vertical respect to the floor. This can be appropriate in hospital environments, or similar. However, in a vehicle, or in other situations in which the person is sitting substantially upright and/or moving in the horizontal plane, magnetic field sensors measuring the component perpendicular to the chest will substantially detect not only the horizontal component of the magnetic field generated by the heart but, also, the changes of the relative direction between the person and the horizontal component of the external fields, such as the Earth's magnetic field. This has special relevance in mobile applications such as, for example, in a vehicle in motion: the angle between the person and the magnetic north can change rapidly (due to, for example, the change of direction of the vehicle in the horizontal plane). Thus, in order to reduce the level of disturbances caused by changes in the orientation of the person with respect to external magnetic fields, it can be preferred to substantially detect (only) the vertical component of the magnetic field generated by the heart. This can be achieved by arranging the measuring direction of the magnetic field sub-sensors in this direction so that at least one core is arranged close to the collarbone of the person under test, where the vertical component of the magnetic fields generated by the heart is maximal. The other sensor should be placed were a “weaker” vertical component of the heart can be sensed, or where a vertical component having an “opposite sense” can be sensed (typically more than 5 cm away from the other sub-sensor).

The differential magnetic field sensor described above can be especially useful for use in a system and method for heart beat rate detection and/or detection of fatigue, as described above. Especially, the sensor can be advantageous for the detection of the heart beat rate of a person in a vehicle. If so, the sensor can advantageously be placed in the seat belt of the driver and/or of other persons in the vehicle, and or in the seat.

Said at least two cores can be placed in the seat-belt of a vehicle, or in the seat of a vehicle, or at least one of said cores can be placed in the seat-belt of a vehicle, and at least another of said cores is placed in the seat of the vehicle (for example, a back rest part of the seat of the vehicle).

A further aspect of the invention relates to the use of this differential magnetic field sensor (or to a plurality thereof) in a system, method and vehicle as described above, or generally for measuring the heart beat rate of a person in a motor vehicle.

### BRIEF DESCRIPTION OF THE DRAWINGS

To complete the description and in order to provide for a better understanding of the invention, a set of drawings is provided. Said drawings form an integral part of the description and illustrate preferred embodiments of the invention, which should not be interpreted as restricting the scope of the invention, but just as an example of how the invention can be embodied. The drawings comprise the following figures:

FIG. 1: Block diagram of the main components of a system in accordance with a preferred embodiment of the invention.

FIGS. 2a and 2b: Schematically illustrate possible positions of the magnetic field sensors. The arrows indicate the sensing axes if uni-axial sensors are used.

FIG. 3: Block diagram of the magnetic field sensor arrangement.

FIG. 4: Block diagram of the signal processing circuitry.

FIG. 5: Flowchart showing a possible algorithm for obtaining data indicative of the heart beat rate FIGS. 6A-6C: Flowcharts showing three appropriate algorithms for fatigue detection.

FIG. 7: Block diagram showing how different approaches for detecting fatigue can be combined to reduce the risk for “false alarms”.

FIG. 8: A schematic representation of a differential magnetic field sensor in accordance with one possible embodiment of the invention.

FIGS. 9A and 9B: A schematic illustration of two alternative arrangements of the secondary windings of such a magnetic field sensor in accordance with an embodiment of the invention.

FIG. 10: Simulation of the output voltage of this type of differential magnetic field sensor.

FIG. 11: Circuit diagram for the differential magnetic field sensor, with associated electronics.

FIG. 12: Schematically illustrates one way of positioning the two cores or “sub-sensors” of the differential magnetic field sensor, for measuring a signal indicative of the heart beat rate of a person sitting in a vehicle.

### DESCRIPTION OF PREFERRED EMBODIMENTS OF THE INVENTION

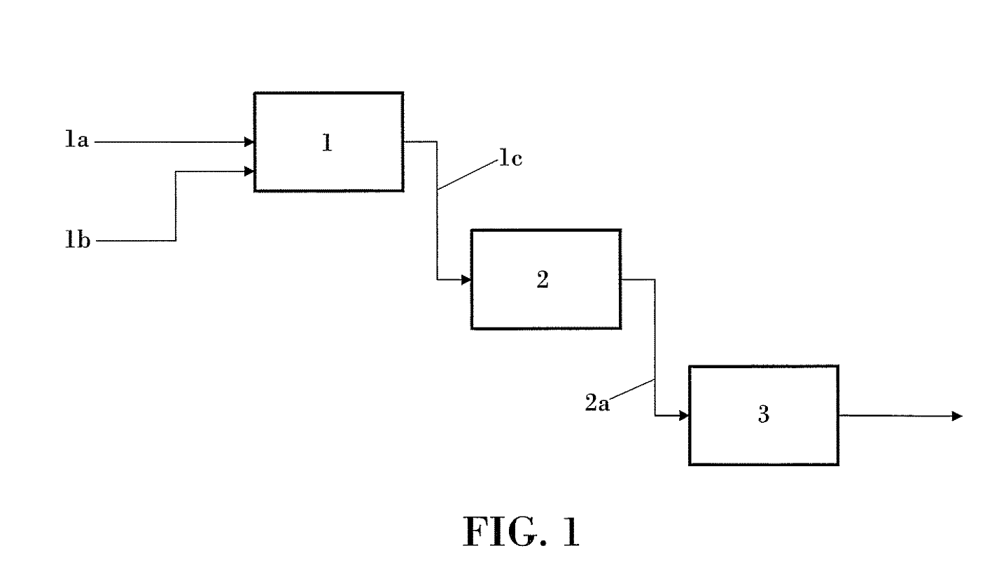

In accordance with a preferred embodiment of the invention shown in FIG. 1, the system comprises a magnetic field sensor module 1 comprising suitably arranged magnetic field sensors, and an electronic signal processing circuitry 2. Also, if the system is a system for fatigue detection, a fatigue detector 3 or somnolence processor can be included.

The magnetic field sensor module 1 comprises, in this embodiment, two uniaxial-fluxgates sensors, (such as FGM-3, produced by Speake & Co), a double regulation power supply, a frequency to voltage converter and a summing (or subtracting) circuit. The magnetic field sensor module detects a signal component 1a related to the magnetic field of the heart, caused by the electrical pulses of the heart, and also a signal component 1b originated by other sources, not related to the heart beat. An output signal 1c from the magnetic field sensor module is supplied to the electronic signal processing circuitry 2, which obtains, from said signal, data indicative of the heart beat rate (for example, data indicating the relative time position of subsequent detected heart beats, or the time between subsequent beats). These data 2a can be used as an input to the fatigue detector. Fatigue detector and signal processing circuitry can obviously be implemented in one single processor module.

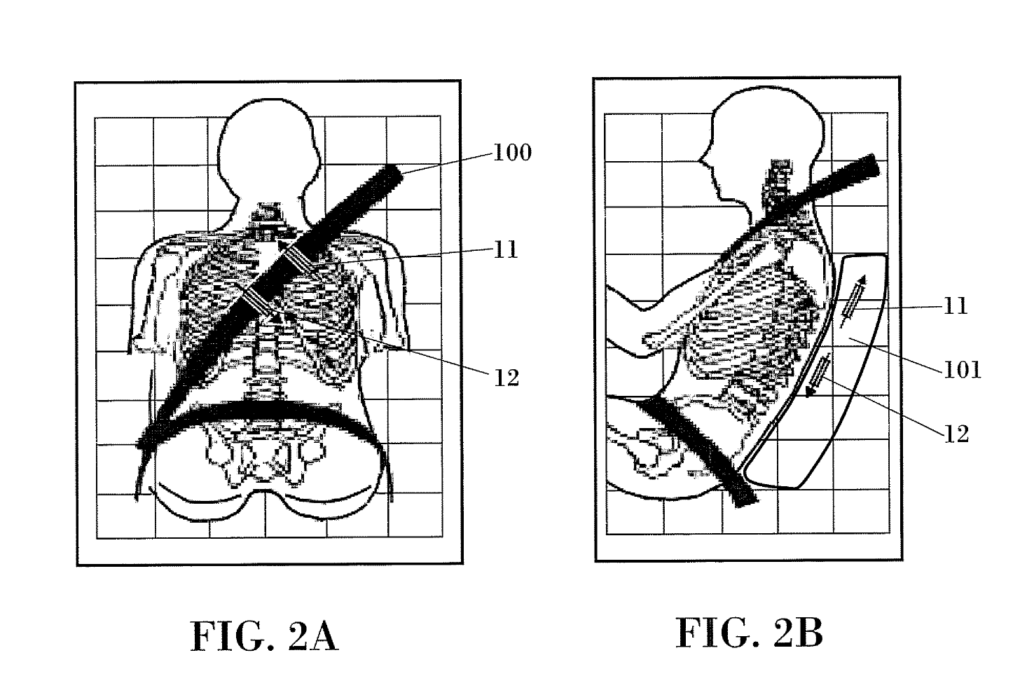

As shown in FIGS. 2a and 2b, the two uni-axial magnetic field sensors 11 and 12 can be placed in the seatbelt 100 (FIG. 2a) or in the seat 101 (FIG. 2b) of a vehicle, with their sensing axes (illustrated by arrows in FIGS. 2a and 2b) parallel to the chest or back, respectively, of the monitored person. If the sensors are placed in the seatbelt, their sensing axes can be arranged perpendicularly to the longitudinal direction of the seatbelt. The sensing axes of the sensors are opposed in FIGS. 2a and 2b (this allows effective subtraction of the sensed signals by using a summing circuit).

This configuration allows good detection of the heart beat related magnetic signal component because the sensing axes are parallel to the main heart magnetic field component.

The sensors can be powered from the battery of the vehicle. In order to reduce supply voltage variations, a double regulation can be used (see FGM-series Magnetic Field Sensors Application Notes, http://www.fatquarterssoftware.com/downloads/fgmapp.pdf), decreasing the voltage from 12-15 V to 9 V first, and then to 5 V.

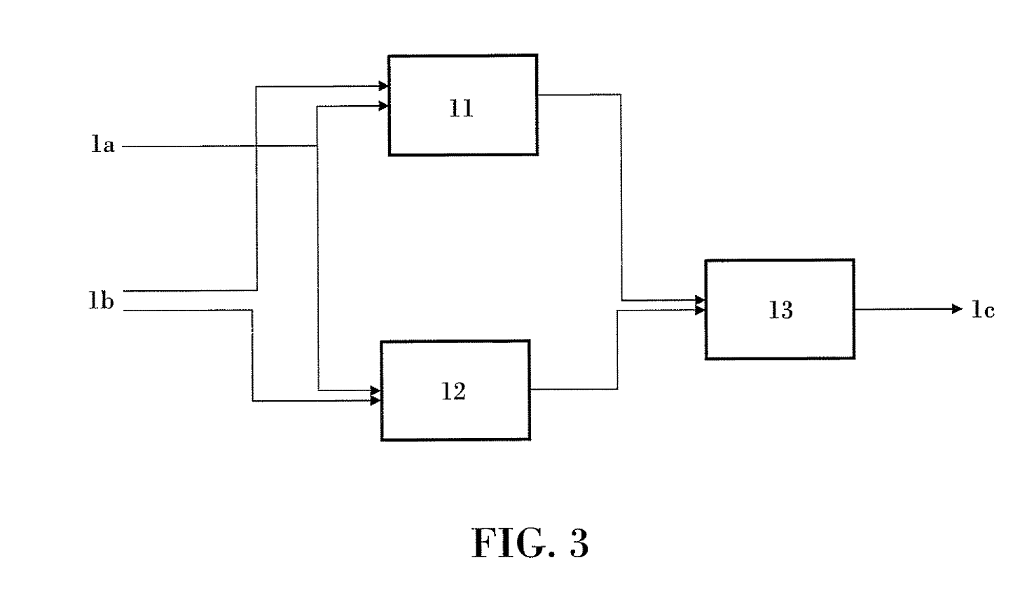

The fluxgate outputs are rectangular pulses whose frequency varies inversely proportional to the magnetic field. The frequency output of every sensor is converted to voltage using a frequency to voltage converter such as LM2907 or equivalent. The two voltages are then put into a summing circuit 13 (which, from a system point of view, can be considered to be included in the electronic signal processing circuitry), as schematically illustrated in FIG. 3 (elements illustrated in FIGS. 1 and 2 are illustrated using the same reference numerals in FIG. 3) (in FIG. 2, the sensing axes of the magnetic field sensors are parallel and directed in opposed senses, whereby a summing circuit 13 can be used for effective subtraction of noise components; if the sensing axis were aligned in the same direction and sense, a subtracting circuit could obviously be used for effective subtraction of the same noise components, that is, of corresponding components in the output signals from the sensors that are due to external magnetic fields not related to the beating of the heart, cf. what has been stated above concerning elimination of non-desired signal components). A variable resistor on one of the inputs makes it possible to adjust the weight of the contribution of each magnetic field sensor, for zeroing the summing (or subtraction) circuit output during calibration. To calibrate the sensors, the arrangement can be placed inside a pair of Helmholtz coils, with the sensing axes direction and the axes of the coils oriented E-W. When a small current passes through the coils, the output of the sum circuit should be zero if both sensors have exactly the same calibration constant. If not, adjusting the variable resistor a zero output can be obtained.

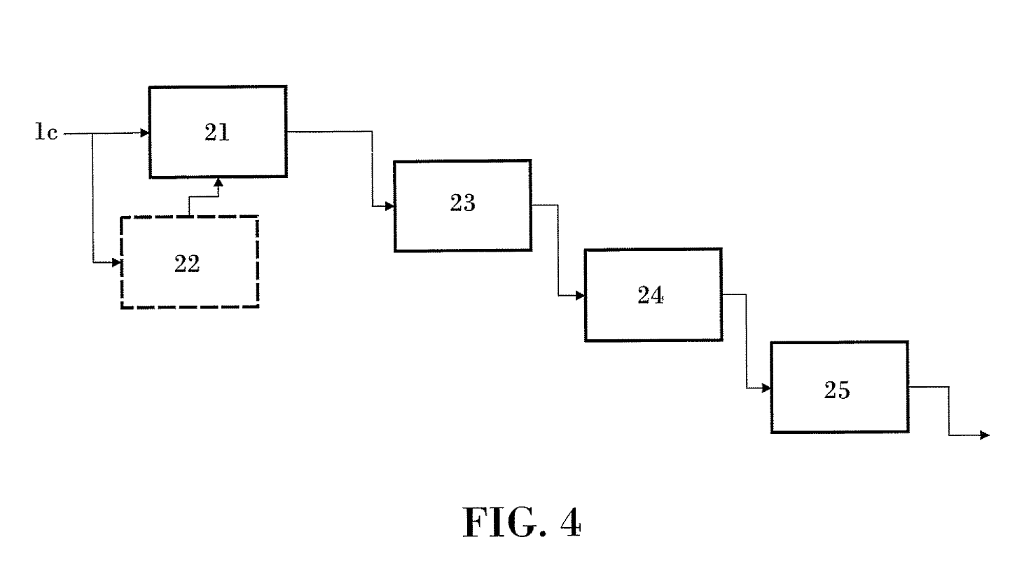

The signal processing circuitry is illustrated in FIG. 4. The output signal 1c from the magnetic field sensor module 1 is supplied to the input of an instrumentation amplifier 21 (such as INA138, from Burr-Brown), with enough gain to obtain a voltage signal with a maximum dynamic range defined by the supply voltage (for example, from 0 to 5V). If the environment where the system is used has a high-power magnetic fluctuation, a derivative circuit 22 can be used, based on an inverting operational amplifier (any standard operational amplifier can be used) in derivative configuration. This derivative circuit can be used to create a virtual reference signal for the instrumentation amplifier in order to compensate this fluctuation.

Afterwards, the signal is fed to a bandpass filter module 23, based on a quad-operational amplifier (such as LM2902). The filter can comprise two stages, with the following characteristics:
Stage 1: high pass, 2^{nd} order, Butterworth active filter with a cutting frequency of 5 Hz and +5 dB of gain.
Stage 2: low pass, 4^{th} order, Butterworth active filter, using two operational amplifiers, with a cutting frequency of 20 Hz and +15 dB of gain.

After amplification and filtering a signal indicative of the heart beat rate is obtained, and can be digitalized with an analogue-to-digital (A/D) converter 24 with, for example, at least 8 bits of resolution. This converter can obviously be integrated in a microprocessor or digital signal processor (DSP). In any case, the digitalized signal is introduced into a microprocessor 25 (or DSP) which processes the signal in order to detect when every beat occurs, and thus produces data directly indicative of the heart beat rate (such as a series of numbers indicating the beat-to-beat time of subsequent beats).

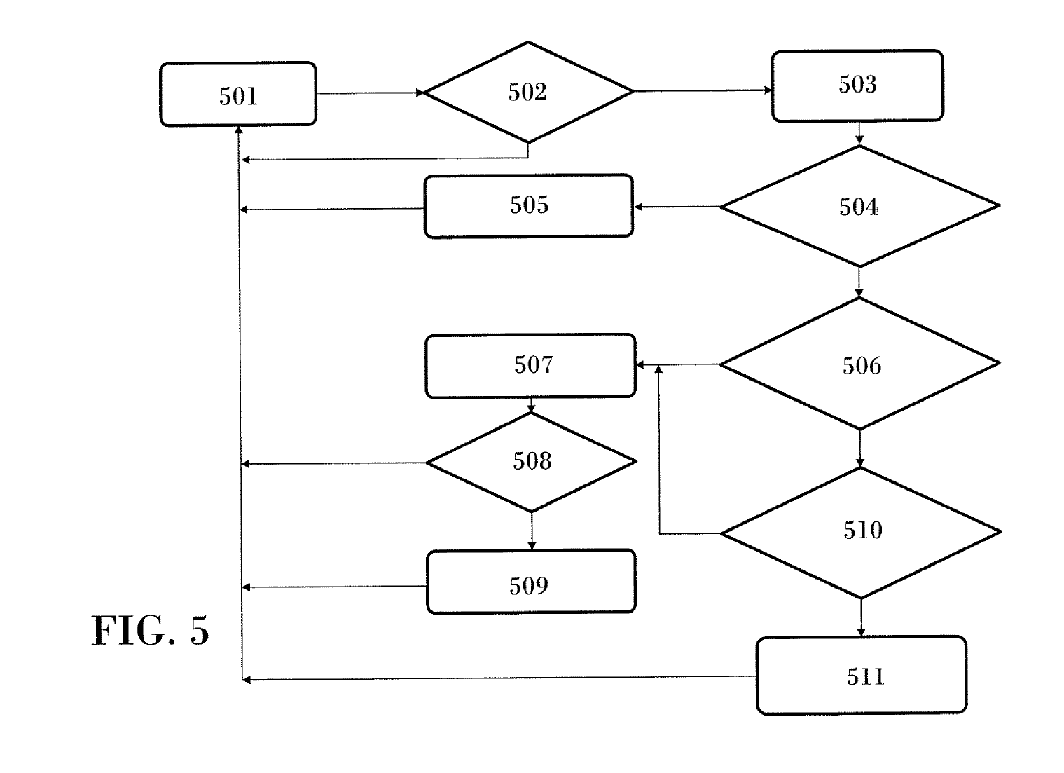

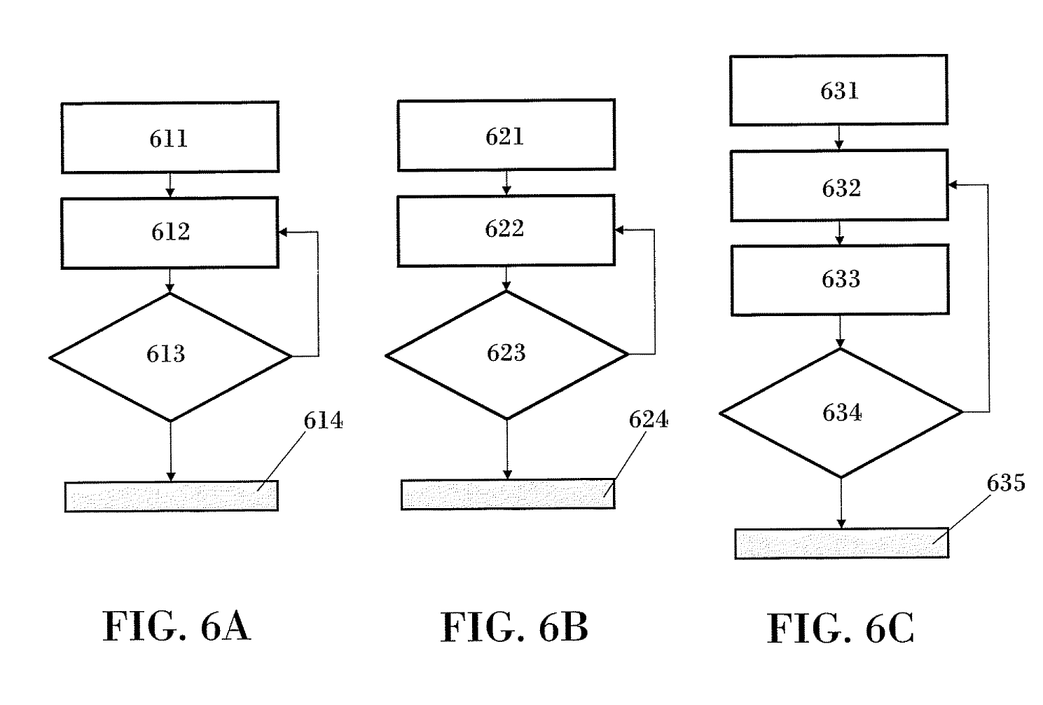

FIG. 5 schematically illustrates how the output signal of the analogue-to-digital converter 25 is sampled (501) by signal processing means associated with the microprocessor. The processing means continue to sample the signal until a (local) maximum is detected (502), which is interpreted as the detection of a new beat (503), whereby the time position of the beat and the magnitude or amplitude of the signal at that moment are registered (503). Next, it is checked (504) whether the magnitude of the “new beat” is much higher than that of the previous beat. If so, it is considered (505) that the previous beat was an invalid beat (due to noise, for example), and the value (magnitude and time position) of the new beat replaces the one of the previous beat. If not, it is checked (506) whether the magnitude of the new beat is similar to the magnitude of the previous beat. If it is not similar, it is considered (507) that the new beat is a “false positive”, that is, that it does not correspond to a beat, and a new sample (501) is obtained. Also, the “false positives” are counted (508) and if they are considered to be too many, the system interprets that it has a bad reference to compare with the new detected beats and resets itself by deleting (509) the information stored as “previous beat”, which is used as a reference for the “false positive” decision.

Now, if the magnitude of the “new beat” is similar to the magnitude of the last detected beat (506), it is checked (510) whether the chronological separation between the new beat and the previous beat is similar to the separation in time between the previous beat and the beat preceding that one. If not, this is once again taken as a “false positive” (507). If yes, the beat is taken as valid beat (511), and the value(s) (such as time position, or delay in time versus the previous beat) replaces the corresponding value(s) of the previous beat, in a FIFO memory buffer (the values corresponding to previous beats are moved towards a “discharge” end of the buffer, and when the buffer is full, every time a new beat is registered, the oldest registered beat is removed). The detection of a valid “new beat” can also trigger the fatigue detector, if the system includes such a detector.

Thus, as can be understood from what has been discussed above, a filtering of “anomalous beats” or “false positives” can be performed both on the basis of the magnitude/amplitude of the detected signal, and of the position in time of the detected “beats”, comparing with data obtained from previous beats and/or with data prestored in the system (relating, for example, to pre-established maximum and minimum beat-to-beat times). For example, if the last “beat-to-beat” distance is less than 80% or more than 120% of the previous “beat-to-beat” distance, this last beat can be considered anomalous and therefore filtered out from the sample (that is, considered to be a “false positive”).

The fatigue detector can be arranged to operate every time a new “valid” beat has been detected and added to the memory buffer or similar, which can be of the FIFO (“First In First Out”) type.

Basically, once a set of data relating to the heart beat rate (such as the beat-to-beat time) has been obtained (for example, once a set of 128 beat-to-beat times has been detected and recorded in the memory buffer), a reference value can be obtained. Next, every time a new piece of data is entered into the memory buffer (whereby the oldest piece of data is removed, if the FIFO type buffer is used), the corresponding current value is counted on the basis of the new set of data. The current value is compared to a predetermined threshold, and if it exceeds said threshold, a fatigue warning event can be triggered (for example, an audible and/or a visible signal can be generated).

Different approaches are schematically illustrated in FIG. 6.

According to a first possible approach, when a buffer (such as a buffer having 128 memory positions for storing 128 subsequently registered beat-to-beat times, in a FIFO manner) is filled for the first time, a “reference value” is calculated (611), this reference value being the average of the beat-to-beat times registered in the buffer at that time. Subsequently, every time a new beat-to-beat time is entered into the buffer (and the “oldest” previous beat-to-beat time is deleted from the buffer content), a “current value” is calculated (612), the current value being the average beat-to-beat time of the new buffer content. Next, it is checked (613) if the current value is more than X % of the reference value, X being typically 110-120. If the current value exceeds this threshold, a fatigue warning event is triggered (614). If the current value is not above said threshold, a new beat-to-beat time value is obtained and stored in the buffer (and the oldest beat-to-beat time is removed from the buffer), and the process is repeated (steps 612-613).

According to a second possible approach, when the buffer is filled for the first time, a reference value is calculated (621), the reference value being the standard deviation of the beat-to-beat times registered in the buffer. Subsequently, every time a new beat-to-beat time is registered in the buffer (and the “oldest” previous beat-to-beat time is deleted from the buffer content), a current value is calculated (622), the current value being the standard deviation of the new buffer content. It is checked (623) if the current value is more than Y % below the reference value, Y being typically in the order of 40. If the current value is more than Y % below the reference value, a fatigue warning event is triggered (624). If not, a new beat-to-beat time value is obtained (and the “oldest” one is removed from the buffer), and the process is repeated (steps 622-623).

According to a third possible approach, when the buffer is filled for the first time, a reference value is calculated (631). This is done by interpolating the buffer content (for example, applying a 2 Hz interpolation), so as to obtain a corresponding continuous signal. To this resulting signal, the Burg algorithm is applied, so as to obtain the spectrum of the signal. Next, the spectral power density is calculated for the LF band (0.04-0.15 Hz) and for the HF band (0.15-0.4 Hz), and by division the LF/HF ratio is obtained. This LF/HF ratio based on the first 128 valid samples is the reference value. Subsequently, each time a new valid beat is detected and the corresponding beat-to-beat time is introduced in the buffer (and the “oldest” previous beat-to-beat time is deleted), a new interpolation is performed so as to obtain a corresponding continuous signal (632), and subsequently the spectral power densities for the LF and HF bands are calculated and the LF/HF ratio is obtained (633); this new LF/HF ration is the current value. Subsequently, it is checked (634) whether the current value is more than Z % below the reference value, Z being typically in the order of 50. If the current value is more than Z % below the reference value, a fatigue warning event is triggered (635). If the current value is not below said threshold, a new beat-to-beat time value is obtained and stored in the buffer (whereby the “oldest” one is removed form the FIFO buffer), and the process is repeated (steps 632-634).

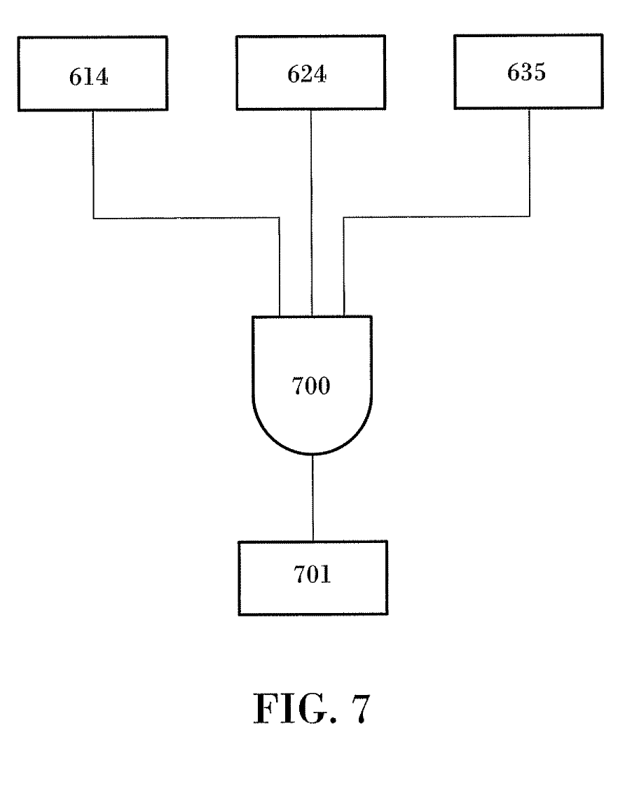

“AND” logic 700 can be used to “combine” two or more of the approaches mentioned above, so as to produce an “effective fatigue warning event” 701 when two or more of said approaches has produced their corresponding “individual” fatigue warning events (614, 624, 635), as schematically illustrated in FIG. 7. If so, no warning signal is sent to the user until said “effective fatigue warning event” is produced.

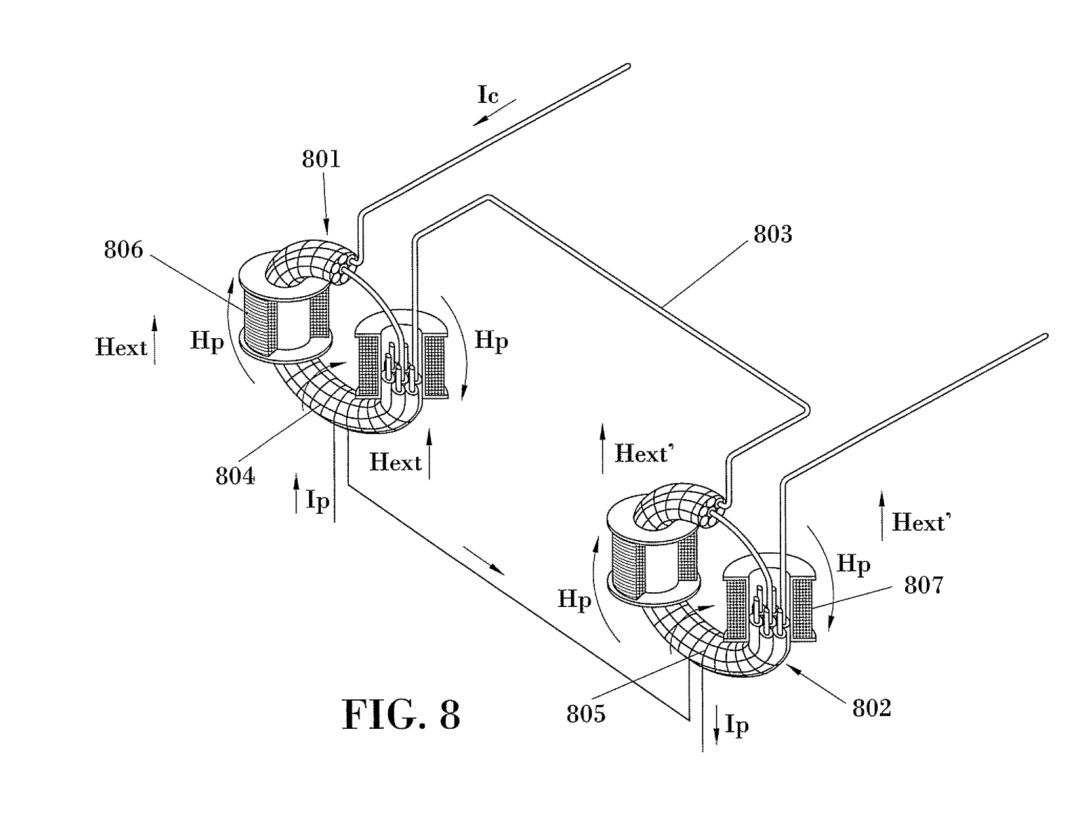

FIG. 8 schematically illustrates a differential magnetic field sensor in accordance with one possible embodiment of the invention, comprising two cores (801, 802) each made up of several turns of an insulated amorphous magnetic wire 803, through which a DC current Ic can be fed, to reduce the noise level, as explained above. The same wire 803 is used for both cores, thus assuring that the DC current through both cores will be the same. Obviously, instead of using one wire, several wires can be used, for example, arranged in parallel.

In accordance with one possible embodiment, the amorphous magnetic wire can have a length in the order of 2 m. A suitable wire is the Co—Fe—Si—B low magnetostriction wire DC2T-100, produced by UNITIKA Ltd, Japan (www.unitika.co.jp), varnished to provide insulation or insulated by passing it trough a plastic tube. The wire can, for each core (801, 802), be wound in a suitable number of turns (such as 15) around a cylindrical support having a diameter of, for example, 15 mm, thus forming a toroidal core.

Although a differential magnetic field sensor with two cores is described, the sensor can obviously have a larger number of cores, in accordance with the needs and cost considerations involved with a specific application of the sensor (the secondary windings should be arranged so as to provide for the necessary “differential” operation of the sensor, taking into consideration the sense of winding of the primary windings and the way the cores are (to be) arranged during operation).

Primary windings 804, 805 are uniformly and toroidally wound on each core 801, 802, with the same number of turns (for example, 450) for each core and using the same wire, having, for example, a diameter of 0.1 mm. Thus, the primary windings 804 and 805 will be serially connected to ensure that the exciting time varying current Ip (which can have an amplitude in the order of 30 mA) is the same for all the cores, both in magnitude and phase.

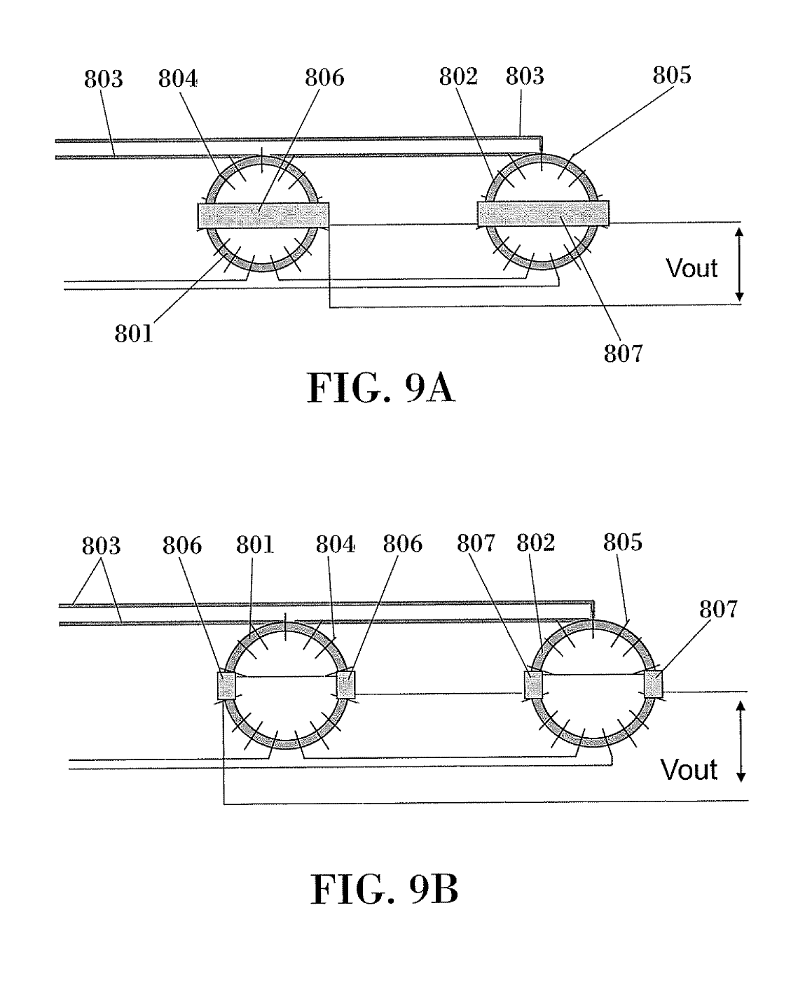

Next, for each core, at least one secondary winding (806, 807) is provided around each core, either surrounding the entire core (that is, extending over the entire “diameter” of the core) or around a “section” of the core, as illustrated in FIG. 8 (cf. also the description below with reference to FIGS. 9A and 9B).

For each of the cores, the secondary winding(s) have the same number of turns (for example, 200 turns). The axes of these secondary windings (806, 807) correspond to the sensing direction of the “sub-sensor” corresponding to each core. Now, the secondary windings (806, 807) corresponding to the two cores (801, 802) are connected in series but with opposite phase. This will electrically subtract the electromotive force of the two cores and will make it possible, with a suitable arrangement of the cores, to obtain an output signal on output terminals of the wire forming the secondary windings, that represents the contribution of the magnetic field generated by the target source, due to the location of one of the cores closer than the other one to the point where the measured magnetic field component generated by the target source is maximal, or alternatively, located at two points were the measured component of the magnetic field generated by the target source has opposite sense.

If bi-axial differential magnetic field sensors are desired, a second secondary winding can be wound along each core so that the axis of the second secondary winding is, for example, perpendicular to the axis of the first secondary winding.

In FIG. 8, the directions of the external magnetic field Hext for coil 801 and Hext for coil 802 can be observed, as well as the directions of the magnetic field Hp generated by the time varying current through the primary windings.

As stated above, the secondary winding (806, 807) can be performed in different ways, two of which are illustrated in FIGS. 9A and 9B. In FIG. 9A (elements described above with reference to FIG. 8 carry the same reference numerals in FIGS. 9A and 9B), it can be seen how the secondary windings are carried out over a “diameter” of the core, so that each turn of the winding surrounds two “legs” of the core, as illustrated in FIG. 9A. Another option for the secondary windings is to embody it as two coils or windings (each having, for example, 200 turns) around radially opposite portions or “legs” of the core, as illustrated in FIG. 9B. The two coils per core of FIG. 9B, when connected with opposed phases, will perform as the winding illustrated in FIG. 9A, but the amount of wire used for these secondary windings of FIG. 9B will be substantially less than the amount of windings used for the secondary windings of FIG. 9A, assuming that the number of turns is the same. The choice between the two configurations can depend on issues such as the available winding tools (toroidal tools are required for the embodiment of FIG. 9B, whereas standard air core tools can be used for the one of FIG. 9A). From a sensing point of view, both configurations can be considered equivalent.

Another option could be to use one single coil having loops that surround both cores.

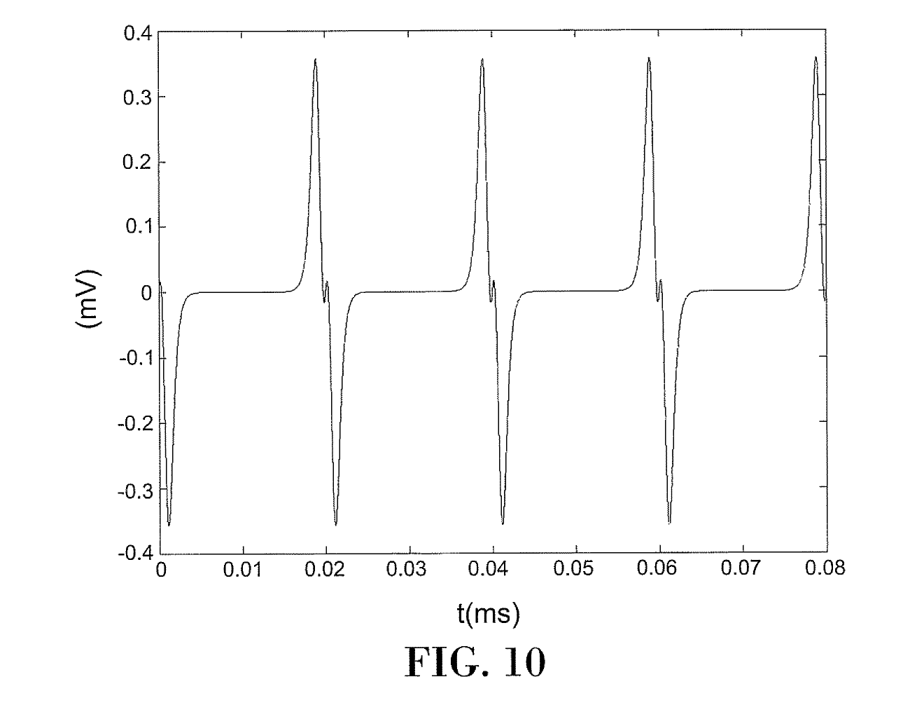

The dual- or multi-core differential magnetic field sensor described above can be driven by standard electronics, using an open loop configuration. The primary coil can be excited with an time varying current Ip (for example, in the order of 30 mA) using a frequency f (for example, 25 kHz), and the output signal can then be the voltage measured over the terminals of the secondary windings, the frequency of which would be 2*f, as schematically illustrated in FIG. 10, which illustrates a simulation of the output voltage (vertical axis) in mV of a differential magnetic field sensor as described above; the horizontal axis is the time axis (in ms). The magnitude of the output voltage is proportional to the difference of the magnetic field (the combination of the magnetic field generated by the target source, and other magnetic fields, including the Earth's magnetic field) at the different cores 801, 802. The small DC current feeding the core (Ip in the order of, for example, 15 mA) reduces the noise by an order of magnitude, increasing the Signal-to-Noise Ratio (SNR).

A second option is to use a closed loop electronic configuration. The electronics used for standard resonant fluxgates magnetometers (such as the ones discussed in the S. Takeuchi and K. Harada reference cited above) can be adapted to be used with this differential configuration. As the primary windings are serially connected, the current passing trough them, Ip, will be the same. Then, if the sensor has been built as described above, both secondary windings will have a similar output voltage, the difference being proportional to the difference of the sensed magnetic field component at the different cores.

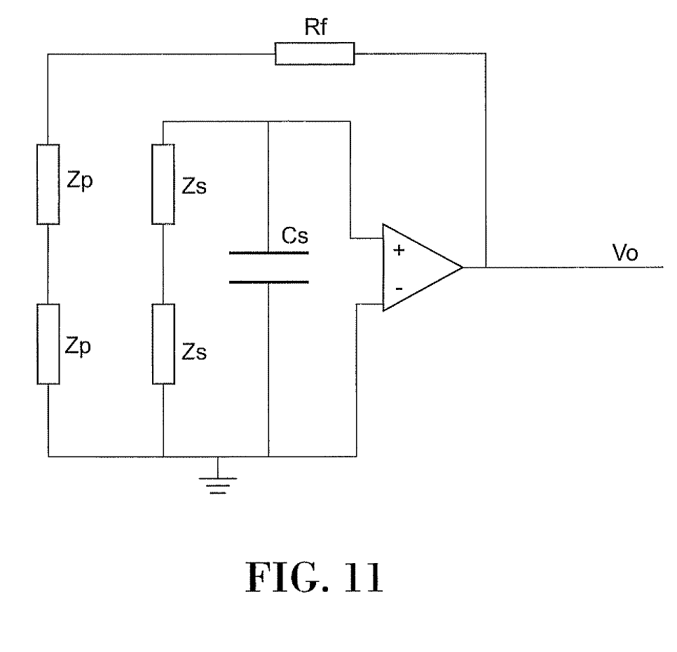

Thus, considering FIG. 11, showing a circuit diagram (in which Zp is the impedance of the primary winding of each core, and Zs the impedance of the secondary winding of each core, Cs a resonance capacitor and Rf a feedback resistor), it can be observed how, when the capacitor Cs is connected in parallel with the output terminals of the sensor (that is, the output terminals of the wire corresponding to the secondary windings), the resonance effect occurs and the resonance frequency can be said to be:

$$f = \frac{1}{2 π \sqrt{{nL}_{s} {C}_{s}}}$$

where n*Ls is the total inductance of the secondary windings (Ls is the inductance of the secondary winding of a single core, and n the number of cores of the sensor). For example, for a device with two cores with an Ls=260 μH secondary coil inductance for each coil and a resonance capacitor (Cs) of 0.1 μF, the resonant frequency is 22 kHz.

The resonant circuit is a common and well-known electronic configuration often used to generate oscillators and high quality frequency filters. The resonance occurs when the impedance of capacitor and inductor are the same and then, any small perturbation on the unstable configuration circuit is amplified and generates a large voltage oscillation, with the mentioned spectral characteristics.

In this application, the initial perturbation is generated by any small magnetic field detected by the differential magnetic field sensor; for example, the Earth's magnetic field could be strong enough to initiate the resonance phenomena.

The output voltage of this resonant circuit is connected to an operational amplifier as suggested in FIG. 11. This amplifier has no direct feedback (just indirect) to the resonant circuit and, thus, it just works as a square signal generator (a comparator of a sinusoidal signal which gives a square output with the same frequency (the resonant frequency shown above) and phase as the original sinusoidal signal).

This squared output signal (Vo) is connected, in positive feedback configuration, to the primary coils (each having an impedance Zp), providing a continuous perturbation of the secondary coils in order to keep the resonance effect infinitely. The feedback resistance Rf converts the output voltage Vo of the operational amplifier into an output current to excite the primary coils. For example, for an output peak voltage Vo=10V, and a feedback resistance Rf=470Ω, a feedback current of 21 mA is obtained. Depending on the core dimensions and the number of turns of the primary windings, the feedback resistance can be calculated to provide a strong enough signal to the secondary windings to maintain the resonant effect.

Therefore, the output voltage Vo is a “rail-to-rail” (that is, with only two levels) output signal oscillating with a frequency proportional to the difference between the magnetic field component sensed at the respective cores. For the described embodiment, the sensitivity is approximately 1 Hz/nT. With a digital frequency or period measuring device having a 0.001% accuracy, changes of 0.1 nT can be detected (an example of a suitable device is the one known as UFDC-1 (http://www.sensorsportal.com)).

If the DC core current Ic is activated, the noise can be reduced below 0.4 nT and the sensitivity and SNR can be sufficiently good to obtain an “MCG” signal strong enough to allow a reliable detection of the heart beat rate or cardiac frequency.

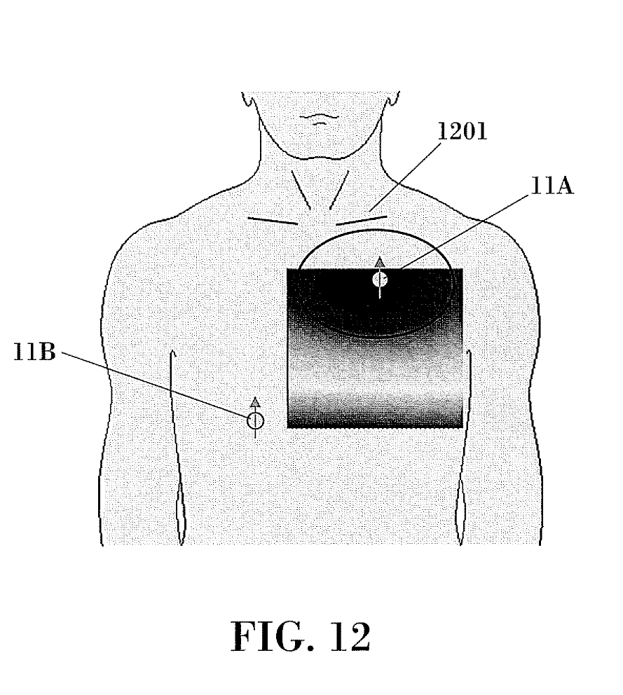

FIG. 12 illustrates one possible way of positioning a magnetic field sensor as the one illustrated in FIG. 8, comprising a first “sub-sensor” 11A corresponding to the core 801 and a second “sub-sensor” 11B corresponding to the core 802. One of the sub-sensors 11A is placed within 10 cm from the collarbone base 1201 of a person and with its sensing axis directed substantially vertically, so as to detect the vertical component of the magnetic field generated by the heart of the person (and, also, the vertical component of the “external sources”). The other “sub-sensor” 11B is positioned in a position further away from the collarbone, at a distance of more than 5 cm from the first sensor, for example, on the other side of the chest and substantially further down. This sub-sensor 11B has its sensing axis aligned with the sensing axis of the first sub-sensor 11A. Thus, most “external” magnetic field sources will affect the sub-sensors in a substantially identical way, and their contributions to the output signal at the secondary winding will thus be cancelled. However, sub-sensor 11A will be subjected to a substantially higher vertical component of the magnetic field generated by the heart than sub-sensor 11B.

The arrangement illustrated in FIG. 12 can be especially useful for the use in vehicles, and the “sub-sensors” making up the differential magnetic field sensor can be implemented in the seat belt (for example, appropriate when measuring the magnetic field in the vicinity of the collarbone) or in the seat (for example, appropriate when measuring the magnetic field in the vicinity of the kidney).

In this text, the term “comprises” and its derivations (such as “comprising”, etc.) should not be understood in an excluding sense, that is, these terms should not be interpreted as excluding the possibility that what is described and defined may include further elements, steps, etc.

On the other hand, the invention is obviously not limited to the specific embodiment(s) described herein, but also encompasses any variations that may be considered by any person skilled in the art (for example, as regards the choice of materials, dimensions, components, configuration, algorithms, etc.), within the general scope of the invention as defined in the claims.

1. A sensor (11, 12) for detecting at least one component of the magnetic field vector at a position in space where the sensor is located, comprising
at least two cores (801, 802), said cores being made up by an insulated amorphous magnetic wire (803), each core comprising a plurality of windings of said amorphous magnetic wire, said amorphous magnetic wire being arranged so that a current can flow through said wire so as to reduce a noise level of the sensor;
for each core, a primary winding (804, 805) arranged in a toroidal manner around said core, said primary winding comprising, for each of the cores, substantially the same number of turns around the core, said primary winding being arranged so that a time varying current can be driven through said primary winding, said primary windings being connected in series so that the time varying current flowing through each primary winding is substantially the same;
for each core, a secondary winding (806, 807) arranged around the core, said secondary windings being connected in series and further being connected to an output terminal of the sensor, for providing an output signal at said output terminal.

2. A sensor according to claim 1, wherein the secondary winding, for at least one of the cores (801, 802), comprises a plurality of loops each of which surrounds the entire core, so that each loop extends over two substantially diametrically opposed portions of the core (FIG. 9A).

3. A sensor according to claim 1, wherein the secondary winding, for at least one of the cores (801, 802), comprises at least two portions, one portion comprising a plurality of loops around a first perimetral portion of the core, and another portion comprising a plurality of loops around a second perimetral portion of the core, angularly displaced along the core with regard to said first perimetral portion (FIG. 9B), and wherein said second perimetral portion is substantially diametrically opposite said first perimeral portion.

4. A sensor according to claim 1 wherein the secondary windings are interconnected so that when the same external magnetic field is applied to said at least two cores oriented in the same manner, the output signal is substantially zero.

5. A sensor according to claim 1, wherein said secondary windings are serially connected so as to provide a differential output signal at least partly indicative of a difference between said component of the magnetic field at one of said cores and at another one of said cores.

6. A sensor according to claim 1, wherein said sensor comprises at least two differentially coupled flux-gate sensors (11A, 11B), each of said flux-gate sensors comprising one of said cores with the corresponding first and secondary windings.

7. A sensor according to claim 1, further comprising electronic circuitry so as to provide a differential output signal indicative of a magnetic field from a target source.

8. A sensor according to claim 7, wherein said electronic circuitry comprises means for producing a DC current in said amorphous magnetic wire and a time varying current in said primary windings.

9. A sensor according to claim 7, wherein said electronic circuitry comprises a resonant closed loop electronic circuitry, and wherein, in said resonant closed loop electronic circuitry, the output terminals of the secondary windings (806, 807; Zs) are coupled to respective input ports of an operational amplifier, in parallel with a resonance capacitor (Cs), whereas the output port of said operational amplifier is connected for feedback to the series connected primary windings (804, 805; Zp) through a feedback resistor (Rf).

10. A sensor according to any of claim 1, arranged to detect the heart beat rate of a person, said sensor being arranged so that at least one of the cores is arranged substantially closer to the collarbone (1201) of the person than at least another of said cores, and wherein at least a first one of said cores is arranged within a distance of 10 cm from said collarbone (1201), and whereas at least a second one of said cores is arranged at a distance of at least 5 cm from the first one of said cores.

11. A sensor according to claim 1, arranged to detect the heart beat rate of a person, said sensor being arranged so that at least one of the cores is arranged substantially closer to the left kidney of the person than at least another of said cores, wherein at least a first one of said cores is arranged within a distance of 10 cm from the left kidney and whereas at least a second one of said cores is arranged at a distance of at least 5 cm from the first one of said cores.

12. A sensor according to claim 1, wherein at least one of said cores is placed in the seatbelt (100) of a vehicle, and at least another of said cores is placed in the seat (101) of the vehicle.

13. A sensor according to claim 12, wherein at least one of said cores is placed in a back rest part of the seat (101) of the vehicle.

14. A system for detecting the heart beat rate of a driver of a vehicle, characterised in that it comprises:
at least one magnetic field sensor (11, 12; 11A+11B) mounted inside the vehicle in a position close to the driver's seat in the vehicle, said at least one magnetic field sensor being arranged for measuring at least one component of the magnetic field vector of the magnetic field generated by the heart of said driver; and
signal processing circuitry (2, 13) arranged to receive an output signal from said at least one magnetic field sensor, and to extract, from said output signal, data indicative of a heart beat rate.

15. A system according to claim 14, wherein said at least one magnetic field sensor (11, 12; 11A+11B) is mounted in a seat belt (100) for the driver in the vehicle.

16. A system according to claim 14, wherein said at least one magnetic field sensor (11, 12; 11A+11B) is mounted in the driver's seat (101).

17. A system according to claim 14, wherein said at least one magnetic field sensor comprises at least two magnetic field sensors (11, 12).

18. A system according to claim 17, wherein said at least two magnetic field sensores are arranged to be placed substantially symmetrically with respect to the driver's heart when the driver is sitting in the vehicle.

19. A system according to claim 17, wherein said at least two magnetic field sensors are arranged at different heights.

20. A system according to claim 17, wherein the signal processing circuitry (2, 13) is arranged to subtract an output signal from one of the magnetic field sensors from an output signal from another of said magnetic field sensor, so as to obtain a resulting signal less influenced by magnetic fields not originated by the heart of the driver.

21. A system according to claim 17, wherein the magnetic field sensors and the signal processing circuitry are arranged so as to produce a subtraction of components of output signals from the magnetic field sensors that are related to external magnetic fields not originated by the heart of the driver, so as to obtain a resulting signal less influenced by magnetic fields not originated by the heart of the driver.

22. A system according to claim 20, wherein the signal processing circuitry (2) is arranged to extract data indicative of a heart beat rate from said resulting signal.

23. A system according to claim 22, wherein said signal processing circuitry comprises fuzzy logic means for extracting said data indicative of a heart beat rate from said resulting signal.

24. A system according to claim 14, wherein at least one of said at least one magnetic field sensors is a magnetic field sensor comprising at least two cores (801, 802) said cores being made up by an insulated amorphous magnetic wire (803), each core comprising a plurality of windings of said amorphous magnetic wire, said amorphous magnetic wire being arranged so that a current can flow through said wire so as to reduce a noise level of the sensor;
for each core, a primary winding (804, 805) arranged in a toroidal manner around said core, said primary winding comprising for each of the cores substantially the same number of turns around the core, said primary winding being arranged so that a time varying current can be driven through said primary winding, said primary windings being connected in series so that the time varying current flowing through each primary winding is substantially the same;
for each core, a secondary winding (806, 807) arranged around the core, said secondary windings being connected in series and further being connected to an output terminal of the sensor for providing an output signal at said output terminal.

25. A system for fatigue detection, for detecting fatigue of a driver of a vehicle, comprising a system according to claim 14, and further comprising a fatigue detector (3) arranged to process the data indicative of a heart beat rate to detect whether said data are indicative of fatigue of a person and, if said data are indicate of fatigue, to produce a fatigue warning event.

26. A vehicle, including a system according to claim 14.

27. A method for detecting the heart beat rate of a driver of a vehicle by measuring at least one component of the magnetic field vector of the magnetic field generated by the heart of the driver, comprising the steps of:
arranging at least one magnetic field sensor (11, 12) inside the vehicle in a position close to the driver's seat in the vehicle, for measuring at least one component of the magnetic field vector of the magnetic field generated by the heart of said driver; and
receiving an output signal from said at least one magnetic field sensor, and extracting, from said output signal, data indicative of a heart beat rate.

28. A method for fatigue detection, for detecting fatigue of a person in a vehicle, comprising the method according to claim 27, and further comprising the steps of processing the data indicative of a heart beat rate to detect whether said data are indicative of fatigue of a person and, if said data are indicate of fatigue, producing a fatigue warning event (614, 624, 635; 701).

29. A method according to claim 28, wherein the processing of the data indicative of a heart rate comprises establishing, based on the data indicative of the heart beat rate, at least one reference value (611, 621, 631) and at least one current value (612, 622, 632, 633), and wherein the fatigue warning event (614, 624, 635; 701) is triggered when at least one current value deviates more than to a predetermined extent from the corresponding reference value.

30. A method according to claim 29, wherein at least one current value and reference value are values indicative of the data indicative of the heart beat rate.

31. A method according to claim 29, wherein at least one current value and reference value are values indicative of the variability of the data indicative of the heart beat rate.

32. A method according to claim 29, wherein at least one current value and reference value are values corresponding to a spectral analysis of the data indicative of the heart beat rate.

33. A method according to claim 32, wherein said current value and reference value correspond to a ratio between a low frequency component and a high frequency component of a curve corresponding to the heart beat rate spectra.

34. A method according to claim 29, wherein said at least one current value and said at least one reference value comprise a plurality of current values and reference values, selected from the group comprising
a current value and a reference value indicative of the data indicative of the heart beat rate;
a current value and a reference value indicative of the variability of the data indicative of the heart beat rate; and
a current value and a reference value corresponding to a spectral analysis of the data indicative of the heart beat rate;
wherein said fatigue warning event (701) is arranged triggered when at least two of the current values deviate more than to a predetermined extent from the corresponding reference values.

35. A method according to claim 27, wherein at least one of said at least one magnetic field sensor is a magnetic field sensor comprising at least two cores (801, 802) said cores being made up by an insulated amorphous magnetic wire (803) each core comprising a plurality of windings of said amorphous magnetic wire, said amorphous magnetic wire being arranged so that a current can flow through said wire so as to reduce a noise level of the sensor;
for each core, a primary winding (804, 805) arranged in a toroidal manner around said core, said primary winding comprising, for each of the cores substantially the same number of turns around the core, said primary winding being arranged so that a time varying current can be driven through said primary winding, said primary windings being connected in series so that the time varying current flowing through each primary winding is substantially the same;
for each core, a secondary winding (806, 807) arranged around the core, said secondary windings being connected in series and further being connected to an output terminal of the sensor, for providing an output signal at said output terminal.

36. Use a magnetic field sensor according to claim 1, in a system according to claim 14.

37. Use of a magnetic field sensor according to claim 1, in a method according to claim 27.

38. Use of a sensor according to claim 1, for measuring the heart beat rate of a person in a motor vehicle.
